(19) United States
(12) Patent Application Publication
(10) Pub. No.: US 2025/0276020 A1
(43) Pub. Date: Sep. 4, 2025

(54) DUAL MACROGLIAL-MICROGLIAL APPROACH TOWARDS THERAPEUTIC CELL REPLACEMENT IN NEURODEGENERATIVE AND NEUROPSYCHIATRIC DISEASE

(71) Applicant: University of Rochester, Rochester, NY (US)

(72) Inventor: Steven A. Goldman, Webster, NY (US)

(73) Assignee: University of Rochester, Rochester, NY (US)

(21) Appl. No.: 18/857,389

(22) PCT Filed: May 4, 2023
(86) PCT No.: PCT/US2023/020962

§ 371 Date: Oct. 16, 2024

Related U.S. Application Data
(60) Provisional application No. 63/338,698, filed on May 5, 2022.

(51) Int. Cl. A61K 35/30

(52) U.S. Cl. CPC A61K 35/30

(57) ABSTRACT

The present application relates to a composition suitable for treating glial-mediated neurodegenerative disease or glial-mediated neuropsychiatric disease comprising a population of isolated human microglial progenitor cells derived from pluripotent stem cells and a population of isolated human macroglial progenitor cells derived from pluripotent stem cells. Also disclosed is a method of treating a glial-mediated neurodegenerative disease or glial-mediated neuropsychiatric disease in a subject. This method includes selecting a subject with a glial-mediated neurodegenerative disease or glial-mediated neuropsychiatric disease and introducing a population of isolated microglial progenitor cells and a population of isolated macroglial progenitor cells into the brain and/or brain stem of the selected subject to at least partially replace cells in the subject's brain with glial-mediated neurodegenerative disease or

### CROSS REFERENCE TO RELATED APPLICATION

This application claims priority to U.S. Provisional Application No. 63/338,698 filed on May 5, 2022. The content of the application is incorporated herein by reference in its entirety.

### GOVERNMENT INTERESTS

This invention was made with government support under DA054534 awarded by the National Institutes of Health. The government has certain rights in the invention.

### FIELD

The present application relates to glial compositions and methods of use thereof.

### BACKGROUND

Neurodegenerative disease is characterized by the progressive loss of nervous system structure and function. The effects of neurodegenerative diseases, such as Huntington's disease and Alzheimer's disease, include cognitive impairment, behavioral changes, dementia, gait disturbances, depression, and, eventually, death. It is now known that in many neurodegenerative diseases, the damage the nervous system is not just limited to neurons themselves. Glial cells, which greatly outnumber neurons in mammalian nervous systems, are often heavily impacted. For example, in Alzheimer's disease, the loss of glial cells, such as astrocytes, and oligodendrocytes, is fundamental for the progression of the disease. As the glial cells fail in their roles of homeostatic regulation and neuroprotection, they leave their corresponding neurons to face higher levels of excitotoxicity and oxidative stress. Dzamba et al., “Glial Cells—The Key Elements of Alzheimer's Disease,” Curr. Alzheimer Res. 13(8):894-911 (2016). Additionally, a hallmark of certain neurodegenerative diseases is a corresponding loss of white matter in the central nervous system, which can involve the loss of several types of glia cells. Brun and E. Englund, “A White Matter Disorder in Dementia of the Alzheimer Type: A Pathoanatomical Study,” Annals of Neurology 19(3):253-262 (1986). For example, Huntington's disease is characterized by the early appearance of white matter demyelination due to the loss of glial cells, which can appear before symptoms arise. Tabrizi et al., “Potential endpoints for clinical trials in premanifest and early Huntington's disease in the TRACK-HD study: analysis of 24 month observational data,” Lancet Neurol. 11:42-53 (2012), and Teo et al., “Structural and Molecular Myelination Deficits Occur Prior to Neuronal Loss in the YAC128 and BACHD Models of Huntinton's disease,” Hum. Mol. Genet. 25:2621-2632 (2016).

In addition to their roles in neurodegenerative disease, the dysfunction or loss of glial cells can be equally impactful in a range of neuropsychiatric disorders. Neuropsychiatric disorders are mental or emotional disorders that arise as a result of underlying conditions affecting a patient's nervous system, such as major depression, bipolar disorder, and schizophrenia. For example, astrocytes plays an important role in the balance of excitatory and inhibitory neurotransmitters in the central nervous system, and astrocytic pathology has been seen in multiple brain regions in patients with depression and mood disorders. Mayegowda and Thomas, “Glial Pathology in Neuropsychiatric Disorders: a Brief Review,” J. Basic Clin. Physiol. Pharmacol. 30(4) (2019). Additionally, developmental and functional deficits in multiple glial cell types has been shown in models of schizophrenia. Liu et al., “Dysregulated Glial Differentiation in Schizophrenia may be Relieved by Suppression of SMAD4 and REST-dependent Signaling,” Cell Rep., 27(13): 3832-43 (2019); Dietz et al., “Glial Cells in Schizophrenia: A Unified Hypothesis,” Lancet Phsyc. 7(3):271-81 (2020).

What is needed is a treatment that can halt or even restore glial cell deficiencies of neurodegenerative and neuropsychiatric diseases. The present application is directed to overcoming these and other deficiencies in the art.

### SUMMARY

The present application relates to a composition suitable for treating glial-mediated human neurodegenerative disease or glial-mediated human neuropsychiatric disease comprising (i) a population of isolated microglial progenitor cells (such as human microglial progenitor cells) derived from pluripotent stem cells and (ii) a population of isolated macroglial progenitor cells (such as human macroglial progenitor cells) derived from pluripotent stem cells. The pluripotent stem cells can be any suitable stem cells. In one embodiment, the pluripotent stem cells are embryonic stem cells. In another embodiment, the pluripotent stem cells are induced pluripotent stem cells.

In the composition, the numerical ratio of the microglial progenitor cells to the macroglial progenitor cells can be about from 1:1,000 to 1,000 to 1. For example, the ratio can range from 1:800 to 800:1, from 1:500 to 500:1, from 1:200 to 200:1, from 1:100 to 100:1, from 1:80 to 80:1, from 1:50 to 50:1, from 1:20 to 20:1, from 1:10 to 10:1, from 1:8 to 8:1, from 1:6 to 6:1, from 1:5 to 5:1, from 1:4 to 4:1, from 1:3 to 3:1, from 1:2 to 2:1, and is about 1:1.

The present application also relates to a method of treating glial-mediated neurodegenerative disease or glial-mediated neuropsychiatric disease in a subject. This method includes selecting a subject with a glial-mediated neurodegenerative disease or a glial-mediated neuropsychiatric disease and introducing or administering a population of isolated microglial progenitor cells derived from pluripotent stem cells and a population of isolated macroglial progenitor cells derived from pluripotent stem cells into the brain and/or brain stem of the selected subject to at least partially replace cells in the subject's brain undergoing glial-mediated neurodegenerative disease or glial-mediated neuropsychiatric disease.

In some embodiments, the subject has a glial-mediated neurodegenerative disease selected from the group consisting of Alzheimer's disease, Lewy body disease, Multisystem atrophy, progressive supernuclear palsy, corticobasal degeneration, and Huntington disease. In one example, the glial-mediated neurodegenerative disease is Alzheimer's disease. In some embodiments, the subject has a glial-mediated neuropsychiatric disease. In one example, the glial-mediated neuropsychiatric disease is schizophrenia. In some embodiments, the selected subject is a human.

In some embodiments, the pluripotent stem cells are embryonic stem cells. In some embodiments, the pluripotent stem cells are induced pluripotent stem cells.

In the method described above, the introducing or administering can be carried out by intraparenchymal, intracallosal, intraventricular, intrathecal, intracerebral, intracisternal, or intravenous transplantation. In some embodiments, the population of isolated microglial progenitor cells and the population of isolated macroglial progenitor cells are from an autologous source. In some embodiments, the population of isolated microglial progenitor cells and the population of isolated macroglial progenitor cells are from an allogenic source.

In some embodiments, the introducing or administering can be carried out by co-engrafting the population of isolated microglial progenitor cells and the population of isolated macroglial progenitor cells.

In some embodiments, the population of isolated microglial progenitor cells and the population of isolated macroglial progenitor cells are introduced separately and sequentially.

In some embodiments, the population of isolated microglial progenitor cells and the population of isolated macroglial progenitor cells are introduced simultaneously.

In some embodiments, the population of isolated microglial progenitor cells and the population of isolated macroglial progenitor cells are introduced together in a composition.

In the method described herein, the numerical ratio of the microglial progenitor cells to the macroglial progenitor cells introduced can be about from 1:1,000 to 1,000 to 1. For example, the ratio can range from 1:800 to 800:1, from 1:500 to 500:1, from 1:200 to 200:1, from 1:100 to 100:1, from 1:80 to 80:1, from 1:50 to 50:1, from 1:20 to 20:1, from 1:10 to 10:1, from 1:8 to 8:1, from 1:6 to 6:1, from 1:5 to 5:1, from 1:4 to 4:1, from 1:3 to 3:1, from 1:2 to 2:1, and is about 1:1. In some embodiments, each of the population of the microglial progenitor cells and the population of the macroglial progenitor cells can be introduced to the subject at a dose of about 1×10² to about 1×10¹⁰. In some embodiments, the population the microglial progenitor or the macroglial progenitor cells can be introduced to the subject at a dose of about 1×10², 1×10³, 1×10⁴, 1×10⁵, 1×10⁶, 1×10⁷, 1×10⁸, 1×10⁹, or 1×10¹⁰.

The ability to replace lost or dysfunctional native microglial and macroglial cells in vivo was previously unknown. This disclosure shows that the treatment of patients having a glial-mediated human neurodegenerative or neuropsychiatric disease with compositions comprising isolated human microglial progenitor cells derived from pluripotent stem cells and isolated macroglial progenitor cells derived from pluripotent stem cells is beneficial in restoring normal glial cell function. Without wishing to be bound by theory, the microglial and macroglial progenitor cells can repopulate brain areas with healthy, mature microglial and macroglial cells because the newly implanted and healthy progenitor cells are younger than, and thus have a proliferative advantage over, native glial cell types.

In a further aspect, the present application provides a kit for treating glial-mediated human neurodegenerative disease or glial-mediated human neuropsychiatric disease. The kit comprises a population of isolated human microglial progenitor cells derived from pluripotent stem cells and a population of isolated human macroglial progenitor cells derived from pluripotent stem cells. The pluripotent stem cells can be any suitable stem cells. In one embodiment, pluripotent stem cells are embryonic stem cells. In another embodiment, the pluripotent stem cells are induced pluripotent stem cells.

### BRIEF DESCRIPTION OF THE DRAWINGS

FIG. 1 shows that human ESC (H9) hematopoietic progenitor cells engrafted as microglia (positive for P2RY12 antibody immunostaining) and outcompeted murine cells to yield a human microglial chimera.

FIG. 2 shows that human hematopoietic and glial progenitor cells (hHPCs and GPCs) can be co-delivered to the neonatal immunodeficient mouse brain, to yield dual chimeras in which most brain microglia (positive for P2RY12 antibody immunostaining) and macroglia (mCherry-tagged) are human, allowing assessment of the effects of the human innate system on transplanted glia.

### DETAILED DESCRIPTION

The present application relates to a composition suitable for treating glial-mediated human neurodegenerative disease or glial-mediated human neuropsychiatric disease comprising a population of isolated human microglial progenitor cells derived from pluripotent stem cells and a population of isolated macroglial progenitor cells derived from pluripotent stem cells.

In some embodiments, the numerical ratio of the microglial progenitor cells to the macroglial progenitor cells is about from 1:1,000 to 1,000 to 1. The numerical ratio refers to the number of cells for one type relative to another type. For example, the ratio can range from 1:800 to 800:1, from 1:500 to 500:1, from 1:200 to 200:1, from 1:100 to 100:1, from 1:80 to 80:1, from 1:50 to 50:1, from 1:20 to 20:1, from 1:10 to 10:1, from 1:8 to 8:1, from 1:6 to 6:1, from 1:5 to 5:1, from 1:4 to 4:1, from 1:3 to 3:1, from 1:2 to 2:1, and is about 1:1.

In some embodiments, each of the population of the microglial progenitor cells and the population of the macroglial progenitor cells can be introduced to the subject at a dose of about 1×10² to about 1×10¹⁰. In some embodiments, the population the microglial progenitor or the macroglial progenitor cells can be introduced to the subject at a dose of about 1×10², 1×10³, 1×10⁴, 1×10⁵, 1×10⁶, 1×10⁷, 1×10⁸, 1×10⁹, or 1×10¹⁰.

As used herein, the term “glial cells” refers to a population of non-neuronal cells that provide support and nutrition, maintain homeostasis, either form myelin or promote myelination, and participate in signal transmission in the nervous system. Glial cells encompasses both microglial cells and macroglial cells, which includes fully differentiated microglial and macroglial cells, as well any microglial and macroglial progenitor cells.

As used herein, the term “microglial cells” refers to a population of glial cells derived from common myeloid progenitor cells. Without wishing to be bound by theory, microglial cells are essentially a specialized macrophage cell for nervous tissue, and derive their name because of their small size compared to other glial cells (see, e.g., Brodal, The Central Nervous System, 4th Ed., Oxford University Press, 2010, Chp. 2, p. 26, which is hereby incorporated by reference in its entirety).

As used herein, the term “microglial progenitor cells” refers to a population of cells that has the potential to develop into mature microglial cells. In some embodiments, microglial progenitor cells cannot develop into mature macroglial cells.

As used herein, the term “macroglial cells” refers to a population glial cells derived from neuroectodermal or neuroepithelial progenitor cells. Macroglial cells include fully differentiated macroglial cells such as, but not limited to, oligodendrocytes and astrocytes, as well as the macroglial progenitor cells themselves.

As used herein, the term “macroglial progenitor cells” refers to a population of cells that has the potential to develop into one of the several types of mature macroglial cells. In some embodiments, macroglial progenitor cells cannot develop into mature microglial cells.

The microglial progenitor cells and macroglial progenitor cells described herein may be derived from any suitable source of pluripotent stem cells, such as, for example and without limitation, human induced pluripotent stem cells (iPSCs) and embryonic stem cells, as described in more detail below.

iPSCs are pluripotent cells that are derived from non-pluripotent cells, such as somatic cells. For example, and without limitation, iPSCs can be derived from tissue, peripheral blood, umbilical cord blood, and bone marrow (see e.g., Cai et al., “Generation of Human Induced Pluripotent Stem Cells from Umbilical Cord Matrix and Amniotic Membrane Mesenchymal Cells,” J. Biol. Chem. 285(15):112227-11234 (2110); Giorgetti et al., “Generation of Induced Pluripotent Stem Cells from Human Cord Blood Cells with only Two Factors: Oct4 and Sox2,” Nat. Protocol. 5(4):811-820 (2010); Streckfuss-Bomeke et al., “Comparative Study of Human-Induced Pluripotent Stem Cells Derived from Bone Marrow Cells, Hair Keratinocytes, and Skin Fibroblasts,” Eur. Heart J. doi:10.1093/eurheartj/ehs203 (Jul. 12, 2012); Hu et al., “Efficient Generation of Transgene-Free Induced Pluripotent Stem Cells from Normal and Neoplastic Bone Marrow and Cord Blood Mononuclear Cells,” Blood doi:10.1182/blood-2010-07-298331 (Feb. 4, 2011); Sommer et al., “Generation of Human Induced Pluripotent Stem Cells from Peripheral Blood using the STEMCCA Lentiviral Vector,” J. Vis. Exp. 68:e4327 doi:10.3791/4327 (2012), which are hereby incorporated by reference in their entirety). The somatic cells are reprogrammed to an embryonic stem cell-like state using genetic manipulation. Exemplary somatic cells suitable for the formation of iPSCs include fibroblasts (see e.g., Streckfuss-Bomeke et al., “Comparative Study of Human-Induced Pluripotent Stem Cells Derived from Bone Marrow Cells, Hair Keratinocytes, and Skin Fibroblasts,” Eur. Heart J. doi:10.1093/eurheartj/ehs203 (2012), which is hereby incorporated by reference in its entirety), such as dermal fibroblasts obtained by a skin sample or biopsy, synoviocytes from synovial tissue, keratinocytes, mature B cells, mature T cells, pancreatic β cells, melanocytes, hepatocytes, foreskin cells, cheek cells, or lung fibroblasts.

Methods of producing induced pluripotent stem cells are known in the art and typically involve expressing a combination of reprogramming factors in a somatic cell. Suitable reprogramming factors that promote and induce iPSC generation include one or more of Oct4, Klf4, Sox2, c-Myc, Nanog, C/EBPα, Esrrb, Lin28, and Nr5a2. In certain embodiments, at least two reprogramming factors are expressed in a somatic cell to successfully reprogram the somatic cell. In other embodiments, at least three reprogramming factors are expressed in a somatic cell to successfully reprogram the somatic cell.

iPSCs may be derived by methods known in the art, including the use integrating viral vectors (e.g., lentiviral vectors, inducible lentiviral vectors, and retroviral vectors), excisable vectors (e.g., transposon and floxed lentiviral vectors), and non-integrating vectors (e.g., adenoviral and plasmid vectors) to deliver the genes that promote cell reprogramming (see e.g., Takahashi and Yamanaka, Cell 126:663-676 (2006); Okita. et al., Nature 448:313-317 (2007); Nakagawa et al., Nat. Biotechnol. 26:101-106 (2007); Takahashi et al., Cell 131:1-12 (2007); Meissner et al. Nat. Biotech. 25:1177-1181 (2007); Yu et al. Science 318:1917-1920 (2007); Park et al. Nature 451:141-146 (2008); and U.S. Patent Application Publication No. 2008/0233610, which are hereby incorporated by reference in their entirety). Other methods for generating IPS cells include those disclosed in WO2007/069666, WO2009/006930, WO2009/006997, WO2009/007852, WO2008/118820, U.S. Patent Application Publication No. 2011/0200568 to Ikeda et al., U.S. Patent Application Publication No 2010/0156778 to Egusa et al., U.S. Patent Application Publication No 2012/0276070 to Musick, and U.S. Patent Application Publication No 2012/0276636 to Nakagawa, Shi et al., Cell Stem Cell 3(5):568-574 (2008), Kim et al., Nature 454:646-650 (2008), Kim et al., Cell 136(3):411-419 (2009), Huangfu et al., Nat. Biotechnol. 26:1269-1275 (2008), Zhao et al., Cell Stem Cell 3:475-479 (2008), Feng et al., Nat. Cell Biol. 11:197-203 (2009), and Hanna et al., Cell 133(2):250-264 (2008) which are hereby incorporated by reference in their entirety.

The methods of iPSC generation described above can be modified to include small molecules that enhance reprogramming efficiency or even substitute for a reprogramming factor. These small molecules include, without limitation, epigenetic modulators such as, the DNA methyltransferase inhibitor 5′-azacytidine, the histone deacetylase inhibitor VPA, and the G9a histone methyltransferase inhibitor BIX-01294 together with BayK8644, an L-type calcium channel agonist. Other small molecule reprogramming factors include those that target signal transduction pathways, such as TGF-β inhibitors and kinase inhibitors (e.g., kenpaullone) (see review by Sommer and Mostoslavsky, “Experimental Approaches for the Generation of Induced Pluripotent Stem Cells,” Stem Cell Res. Ther. 1:26 doi:10.1186/scrt26 (Aug. 10, 2010), which is hereby incorporated by reference in its entirety).

Methods of obtaining highly enriched preparations of macroglial progenitor cells from the iPSCs that are suitable for making the non-human mammal models described herein are disclosed in WO2014/124087 to Goldman and Wang, and Wang et al., “Human iPSC-Derived Oligodendrocyte Progenitors Can Myelinate and Rescue a Mouse Model of Congenital Hypomyelination,” Cell Stem Cell 12(2):252-264 (2013), which are hereby incorporated by reference in their entirety.

In another embodiment of the present application, the glial progenitor cells are derived from embryonic stem cells. Embryonic stem cells are derived from totipotent cells of the early mammalian embryo and are capable of unlimited, undifferentiated proliferation in vitro. As used herein, the term “embryonic stem cells” refer to a cells isolated from an embryo, placenta, or umbilical cord, or an immortalized version of such a cells, i.e., an embryonic stem cell line. Suitable embryonic stem cell lines include, without limitation, lines WA-01 (H1), WA-07, WA-09 (H9), WA-13, and WA-14 (H14) (Thomson et al., “Embryonic Stem Cell Lines Derived from Human Blastocytes,” Science 282 (5391): 1145-47 (1998) and U.S. Pat. No. 7,029,913 to Thomson et al., which are hereby incorporated by reference in their entirety). Other suitable embryonic stem cell lines include the HAD-C100 cell line (Tannenbaum et al., “Derivation of Xeno-free and GMP-grade Human Embryonic Stem Cells—Platforms for Future Clinical Applications,” PLoS One 7(6):e35325 (2012), which is hereby incorporated by reference in its entirety, the WIBR4, WIBR5, WIBR6 cell lines (Lengner et al., “Derivation of Pre-x Inactivation Human Embryonic Stem Cell Line in Physiological Oxygen Conditions,” Cell 141(5):872-83 (2010), which is hereby incorporated by reference in its entirety), and the human embryonic stem cell lines (HUES) lines 1-17 (Cowan et al., “Derivation of Embryonic Stem-Cell Lines from Human Blastocytes,” N. Engl. J. Med. 350:1353-56 (2004), which is hereby incorporated by reference in its entirety).

Human embryonic stem cells provide a virtually unlimited source of clonal/genetically modified cells potentially useful for tissue replacement therapies. Methods of obtaining highly enriched preparations of glial progenitor cells from embryonic cells that are suitable for making the non-human mammal model of the present disclosure are described herein as disclosed in Wang et al., “Human iPSC-derived oligodendrocyte progenitor cells can myelinate and rescue a mouse model of congenital hypomyelination,” Cell Stem Cell 12:252-264 (2013), which is hereby incorporated by reference in its entirety.

Briefly, microglial and macroglial progenitor cells are derived from a pluripotent population of cells, i.e., iPSCs or embryonic stem cells, using protocols that directs the pluripotent cells through serial stages of glial progenitor cell differentiation. Each stage of lineage restriction is characterized and identified by the expression of certain cell proteins.

For microglial cell formation, the general procedure is outlined by McQuade et al., “Development and Validation of a Simplified Method to Generate Human Microglia from Pluripotent Stem Cells,” Mol. Neurodenger. 13:67 (2018), which is hereby incorporated by reference in its entirety. This process involves differentiation of iPSCs to CD43+ primitive hematopeietic progenitor cells (HPCs). Feeder-free iPSCs that have been expanded in TeSR-E8 media are passaged with ReLeaSR (STEMCELL technologies) into mTeSR E8 medium with 0.5 μM Thiazovivin onto matrigel coated (1 mg/mL) 6-well plates. Small aggregates of ˜100 cells each are plated at 10-20 aggregates per cm2. When approximately two 100 cell colonies per cm2 have been achieved, the TeSR-E8 medium is replaced with medium A (Basal medium plus Supplement A at 1:200 dilution, 2 mL per well of a 6-well). On day 2 (48 h after original media change), do not fully change media, but rather replace 50% medium A, 1 mL per well of a 6-well. On day 3, carefully remove all media by tilting the plate to one side and aspirating from the edge. Then add 2 mL/well medium B (Basal medium plus supplement B at 1:200). Without removing media, supplement with 1 mL/well of medium B on days 5, 7, 9. On day 10 and again on day 12, non-adherent cells may be collected. To maintain purity, do not wash cells off the well, but merely remove medium with non-adherent cells carefully and centrifuge 300×G 5 min. After centrifugation, replace conditioned medium back to each well and add 1 mL fresh medium B if further collection on day 12 will be completed.

The population of microglial progenitor cells used in carrying out the method of the present application comprises at least about 80% microglial progenitor cells, including, for example, about 80%, 85%, 90%, 95%, 96%, 97%, 98%, 99%, 100% microglial cells. The selected preparation of microglial progenitor cells can be relatively devoid (e.g., containing less than 20, 15, 10, 9, 8, 7, 6, 5, 4, 3, 2, or 1%) of other cells types such as neurons and neuronal progenitor cells. Optionally, the cell population can be a substantially pure populations of microglial progenitor cells.

For macroglial cell formation, stage 1 of this process involves culturing the pluripotent cell population under conditions effective to induce embryoid body formation. As described herein, the pluripotent cell population may be maintained in co-culture with other cells, such as embryonic fibroblasts, in an embryonic stem cell (ESC) media (e.g., DMEM/F12 containing a suitable serum replacement and bFGF). The pluripotent cells are passaged before reaching 100% confluence, e.g., 80% confluence, when colonies are approximately 250-300 μm in diameter. The pluripotential state of the cells is readily assessed using markers to SSEA4, TRA-1-60, OCT-4, NANOG, and/or SOX2.

To generate embryoid bodies (EBs) (Stage 2), which are complex three-dimensional cell aggregates of pluripotent stem cells, pluripotent cell cultures are dissociated once they achieved ˜80% confluence with colony diameters at or around 250-300 μm. The EBs are initially cultured in suspension in ESC media without bFGF, and then switched to neural induction medium supplemented with bFGF and heparin. To induce neuroepithelial differentiation (Stage 3) EBs are plated and cultured in neural induction medium supplemented with bFGF, heparin, laminin, then switched to neural induction media supplemented with retinoic acid. Neuroepithelial differentiation is assessed by the co-expression of PAX6 and SOX1, which characterize central neural stem and progenitor cells.

To induce pre-oligodendrocyte progenitor cell (“pre-OPCs”) differentiation, neuroepithelial cell colonies are cultured in the presence of additional factors including retinoic acid, B27 supplement, and a sonic hedgehog (shh) agonist (e.g., purmophamine). The appearance of pre-OPC colonies is assessed by the presence of OLIG2 and/or NKX2.2 expression. While both OLIG2 and NKX2.2 are expressed by central oligodendrocyte progenitor cells, NKX2.2 is a more specific indicator of oligodendroglial differentiation. Accordingly, an early pre-oligodendrocyte progenitor cell stage is marked by OLIG⁺/NKX2.2⁻ cell colonies. OLIG⁺/NKX2.2⁻ early pre-OPCs are differentiated into later-stage OLIG⁺/NKX2.2⁺ pre-OPCs by replacing retinoic acid with bFGF. At the end of Stage 5, a significant percentage of the cells are pre-OPCs as indicated by OLIG2⁺/NKX2.2⁺ expression profile.

Pre-OPCs are further differentiated into bipotential glial progenitor cells by culture in glial induction media supplemented with growth factors such as triiodothyronine (T3), neurotrophin 3 (NT3), insulin growth factor (IGF-1), and platelet-derived growth factor-AA (PDGF-AA) (Stage 6). These culture conditions can be extended for 3-4 months or longer to maximize the production of myelinogenic glial progenitor cells when desired. Cell preparations suitable for transplantation into an appropriate subject are identified as containing PDGFRα⁺ glial progenitor cells.

The population of macroglial progenitor cells used in carrying out the method of the present application comprises at least about 80% macroglial progenitor cells, including, for example, about 80%, 85%, 90%, 95%, 96%, 97%, 98%, 99%, 100% macroglial cells. The selected preparation of macroglial progenitor cells can be relatively devoid (e.g., containing less than 20, 15, 10, 9, 8, 7, 6, 5, 4, 3, 2, or 1%) of other cells types such as neurons and neuronal progenitor cells. Optionally, the cell population can be a substantially pure populations of macroglial progenitor cells.

The present application also relates to a method of treating glial-mediated neurodegenerative disease or glial-mediated neuropsychiatric disease in a subject. This method includes selecting a subject with a glial-mediated neurodegenerative disease or a glial-mediated neuropsychiatric disease and introducing a population of isolated microglial progenitor cells derived from pluripotent stem cells and a population of isolated macroglial progenitor cells derived from pluripotent stem cells into the brain and/or brain stem of the selected subject to at least partially replace cells in the subject's brain undergoing glial-mediated neurodegenerative disease or glial-mediated neuropsychiatric disease.

In some embodiments, the subject being treated in accordance with the method of the present application is an adult afflicted with a glial-mediated neurodegenerative pathology. Glial-mediated neurodegenerative pathologies include, but are not limited to, Alzheimer's disease, Lewy body disease, multisystem atrophy, progressive supernuclear palsy, corticobasal degeneration, and Huntington disease.

In some embodiments, the subject being treated in accordance with the method of the present application is an adult afflicted with a glial-mediated neuropsychiatric pathologies. Glial-mediated neuropsychiatric pathologies include, but are not limited to, major depression, bipolar disorder, and schizophrenia.

As used herein, “treating” or “treatment” refers to any indication of success in amelioration of an injury, pathology, or condition, including any objective or subjective parameter such as abatement; remission; diminishing of symptoms or making the injury, pathology, or condition more tolerable to the patient; slowing the rate of degeneration or decline; making the final point of degeneration less debilitating; or improving a subject's physical or mental well-being. The treatment or amelioration of symptoms can be based on objective or subjective parameters; including the results of a physical examination, neurological examination, and/or psychiatric evaluation. “Treating” includes the administration of glial progenitor cells to prevent or delay, to alleviate, or to arrest or inhibit development of the symptoms or conditions associated with the disease, condition or disorder. “Therapeutic effect” refers to the reduction, elimination, or prevention of the disease, symptoms of the disease, or side effects of a disease, condition or disorder in the subject. Treatment may be prophylactic (to prevent or delay the onset or worsening of the disease, condition or disorder, or to prevent the manifestation of clinical or subclinical symptoms thereof) or therapeutic suppression or alleviation of symptoms after the manifestation of the disease, condition or disorder.

As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments, a subject is a non-human disease model. In some embodiments, a human subject is an adult, adolescent, or pediatric subject. In some embodiments, a subject is suffering from a disease, disorder or condition, e.g., a disease, disorder or condition that can be treated as provided herein. In some embodiments, a subject is suffering from a disease, disorder or condition associated with deficient or dysfunctional myelin. In some embodiments, a subject is susceptible to a disease, disorder, or condition. In some embodiments, a susceptible subject is predisposed to and/or shows an increased risk (as compared to the average risk observed in a reference subject or population) of developing a disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a human patient. In some embodiments, a subject is an individual to whom diagnosis and/or therapy is and/or has been administered.

The term “patient” is used herein to describe an animal, preferably a human, to whom treatment, including prophylactic treatment, with the cells according to the present invention, is provided. The term “donor” is used to describe an individual (animal, including a human) who or which donates cells or tissue for use in a patient.

As used herein, the term “white matter” relates to a component of the central nervous system, in the brain and superficial spinal cord, which consists mostly of glial cells and myelinated axons that transmit signals from one region of the cerebrum to another and between the cerebrum and lower brain centers.

The term “autologous” refers to any material derived from the same subject or individual to which it is later to be re-introduced. For example, the autologous cell therapy method described herein involves collection of glial cells, or progenitors thereof from a donor, e.g., a patient, which are then engineered to express, e.g., a transgene, and then administered back to the same donor, e.g., patient.

The term “heterologous” refers to any material (e.g., cells or tissue scaffold) derived from a different subject or individual. As used herein, “heterologous” or “non-endogenous” or “exogenous” also refers to any material (e.g., gene, protein, compound, molecule, cell, or tissue or tissue component) or activity that is not native to a host cell or a host subject, or is any gene, protein, compound, molecule, cell, tissue or tissue component, or activity native to a host or host cell but has been altered or mutated such that the structure, activity or both is different as between the native and mutated versions.

The term “allogeneic” refers to any material (e.g., cells or tissue) derived from one individual which is then introduced to another individual of the same species, e.g., allogeneic cell transplantation. For example, cells may be obtained from a first subject, modified ex vivo according to the methods described herein and then administered to a second subject in order to treat a disease. In such embodiments, the cells administered to the subject are allogeneic and heterologous cells. The term “xenogenic” refers to any material (e.g., cells or tissue) derived from an individual of a different species. The term “isogenic” refers to any materials (e.g., cells or tissue) characterized by essentially identical genes.

As used herein, the term “therapeutically effective amount” refers to an amount that produces the desired therapeutic effect for which it is administered. In some embodiments, the term refers to an amount that is sufficient, when administered to a population suffering from or susceptible to a disease, disorder or condition in accordance with a therapeutic dosing regimen, to treat the disease, disorder or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment.

The microglial and macroglial progenitor cells may be introduced into the subject needing treatment of a neurodegenerative pathology by a variety of know techniques. These include, but are not limited to, injection, deposition, and grafting as described herein.

In one embodiment, the microglial and macroglial progenitor cells are transplanted bilaterally into multiple sites of the subject, as described U.S. Pat. No. 7,524,491 to Goldman, Windrem et al., “Neonatal Chimerization With Human Glial Progenitor Cells Can Both Remyelinate and Rescue the Otherwise Lethally Hypomyelinated Shiverer Mouse,” Cell Stem Cell 2:553-565 (2008), Han et al., “Forebrain Engraftment by Human Glial Progenitor Cells Enhances Synaptic Plasticity and Learning Adult Mice,” Cell Stem Cell 12:342-353 (2013), and Wang et al., “Human iPSCs-Derived Oligodendrocyte Progenitor Cells Can Myelinate and Rescue a Mouse Model of Congenital Hypomyelination,” Cell Stem Cell 12:252-264 (2013), which are hereby incorporated by reference in their entirety). Methods for transplanting nerve tissues and cells into host brains are described by Bjorklund and Stenevi (eds), Neural Grafting in the Mammalian CNS, Ch. 3-8, Elsevier, Amsterdam (1985); U.S. Pat. No. 5,082,670 to Gage et al.; and U.S. Pat. No. 6,497,872 to Weiss et al., which are hereby incorporated by reference in their entirety. Typical procedures include intraparenchymal, intracallosal, intraventricular, intrathecal, and intravenous transplantation.

Intraparenchymal transplantation is achieved by injection or deposition of tissue within the host brain so as to be apposed to the brain parenchyma at the time of transplantation. The two main procedures for intraparenchymal transplantation are: 1) injecting the donor cells within the host brain parenchyma or 2) preparing a cavity by surgical means to expose the host brain parenchyma and then depositing the graft into the cavity (Bjorklund and Stenevi (eds), Neural Grafting in the Mammalian CNS, Ch. 3, Elsevier, Amsterdam (1985), which is hereby incorporated by reference in its entirety). Both methods provide parenchymal apposition between the donor cells and host brain tissue at the time of grafting, and both facilitate anatomical integration between the graft and host brain tissue. This is of importance if it is required that the donor cells become an integral part of the host brain and survive for the life of the host.

Microglial and macroglial progenitor cells can also be delivered intracallosally as described in U.S. Patent Application Publication No. 20030223972 to Goldman, which is hereby incorporated by reference in its entirety. The microglial and macroglial progenitor cells can also be delivered directly to the forebrain subcortex, specifically into the anterior and posterior anlagen of the corpus callosum. Microglial and macroglial progenitor cells can also be delivered to the cerebellar peduncle white matter to gain access to the major cerebellar and brainstem tracts. Microglial and macroglial progenitor cells can also be delivered to the spinal cord.

Alternatively, the cells may be placed in a ventricle, e.g., a cerebral ventricle. Grafting cells in the ventricle may be accomplished by injection of the donor cells or by growing the cells in a substrate such as 30% collagen to form a plug of solid tissue which may then be implanted into the ventricle to prevent dislocation of the graft cells. For subdural grafting, the cells may be injected around the surface of the brain after making a slit in the dura.

Suitable techniques for cell delivery are described supra. In one embodiment, said preparation of microglial and macroglial progenitor cells is administered to the striatum, forebrain, brain stem, and/or cerebellum of the subject.

Delivery of the cells to the subject can include either a single step or a multiple step injection directly into the nervous system. For localized disorders such as demyelination of the optic nerve, a single injection can be used. For widespread disorders, multiple injections sites can be performed to optimize treatment. Injection is optionally directed into areas of the central nervous system such as white matter tracts like the corpus callosum (e.g., into the anterior and posterior anlagen), dorsal columns, cerebellar peduncles, cerebral peduncles. Such injections can be made unilaterally or bilaterally using precise localization methods such as stereotaxic surgery, optionally with accompanying imaging methods (e.g., high resolution MRI imaging). One of skill in the art recognizes that brain regions vary across species; however, one of skill in the art also recognizes comparable brain regions across mammalian species.

The cellular transplants are optionally injected as dissociated cells but can also be provided by local placement of non-dissociated cells. In either case, the cellular transplants optionally comprise an acceptable solution. Such acceptable solutions include solutions that avoid undesirable biological activities and contamination. Suitable solutions include an appropriate amount of a pharmaceutically-acceptable salt to render the formulation isotonic. Examples of the pharmaceutically-acceptable solutions include, but are not limited to, saline, Ringer's solution, dextrose solution, and culture media. The pH of the solution is preferably from about 5 to about 8, and more preferably from about 7 to about 7.5.

The injection of the dissociated cellular transplant can be a streaming injection made across the entry path, the exit path, or both the entry and exit paths of the injection device (e.g., a cannula, a needle, or a tube). Automation can be used to provide a uniform entry and exit speed and an injection speed and volume.

The number of microglial and macroglial progenitor cells administered to the subject can each range from about 10²-10⁸ at each administration (e.g., injection site), depending on the size and species of the recipient, and the volume of tissue requiring cell replacement. Single administration (e.g., injection) doses can span ranges of 10³-10⁵, 10⁴-10⁷, and 10⁵-10⁸ cells, or any amount in total for a transplant recipient patient.

Since the CNS is an immunologically privileged site, administered cells, including xenogeneic, can survive and, optionally, no immunosuppressant drugs or a typical regimen of immunosuppressant agents are used in the treatment methods. However, optionally, an immunosuppressant agent may also be administered to the subject. Immunosuppressant agents and their dosing regimens are known to one of skill in the art and include such agents as Azathioprine, Azathioprine Sodium, Cyclosporine, Daltroban, Gusperimus Trihydrochloride, Sirolimus, and Tacrolimus. Dosages ranges and duration of the regimen can be varied with the disorder being treated; the extent of rejection; the activity of the specific immunosuppressant employed; the age, body weight, general health, sex and diet of the subject; the time of administration; the route of administration; the rate of excretion of the specific immunosuppressant employed; the duration and frequency of the treatment; and drugs used in combination. One of skill in the art can determine acceptable dosages for and duration of immunosuppression. The dosage regimen can be adjusted by the individual physician in the event of any contraindications or change in the subject's status.

In one embodiment, one or more immunosuppressant agents are administered to the subject starting at 10 weeks prior to cell administration. In one embodiment, the one or more immunosuppressant agents are administered to the subject starting at 9 weeks, 8 weeks, 7 weeks, 6 weeks, 5 weeks, 4 weeks, 3 weeks, 2 weeks, 1 week, 7 days, 6 days, 5 days, 4 days, 3 days, 2 days, 1 day, <24 hours prior to cell administration. In one embodiment, one or more immunosuppressant agents are administered to the subject starting on the day of cell administration and continuing for 1, 2, 3, 4, 5, 6, 7, 8, 9, 10, 11, 12 months post administration. In one embodiment, the one or more immunosuppressant agents are administered to the subject for >1 year following administration.

Suitable subjects for treatment in accordance with the methods described herein include any mammalian subject afflicted with a glial-mediated neurodegenerative disease or glial-mediated neuropsychiatric disease. Exemplary mammalian subjects include humans, mice, rats, guinea pigs, and other small rodents, dogs, cats, sheep, goats, and monkeys. In one embodiment, the subject is human.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Other meanings of “about” may be apparent from the context, such as rounding off, so, for example “about 1” may also mean from 0.5 to 1.4. Alternatively, the term “about” refers to within an acceptable standard error of the mean, when considered by one of ordinary skill in the art.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

### Pharmaceutical Composition

The present application further provides a composition comprising (i) a carrier and (ii) one or more cells described herein or cells (such as progeny cells) derived therefrom. The composition can be a pharmaceutical composition where the carrier is a pharmaceutically acceptable carrier.

A composition for pharmaceutical use, e.g., an implant with cells and/or factors, can include, depending on the formulation desired, pharmaceutically acceptable, non-toxic carriers of diluents, which are defined as vehicles commonly used to formulate pharmaceutical compositions for animal or human administration. The diluent can be selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide (e.g., a growth factor), the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids. Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see Langer, Science 249:1527-1533 (1990).

The pharmaceutical composition described herein, e.g., progenitor cells alone or in combinations with various factors, can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50.

Data obtained from cell culture and/or animal studies can be used in formulating a range of dosages for humans. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxin, which may be present during the synthesis or purification process. Compositions for parental administration are also sterile, substantially isotonic and made under GMP conditions.

The effective amount of a therapeutic composition to be given to a particular patient will depend on a variety of factors, several of which will differ from patient to patient. A competent clinician will be able to determine an effective amount of a therapeutic agent to administer to a patient to halt or reverse the progression of the disease condition as required. Utilizing animal data, and other information available for the agent, a clinician can determine the maximum safe dose for an individual, depending on the route of administration. For instance, an intravenously administered dose may be more than a locally administered dose, given the greater body of fluid into which the therapeutic composition is being administered. Similarly, compositions that are rapidly cleared from the body may be administered at higher doses, or in repeated doses, in order to maintain a therapeutic concentration. Utilizing ordinary skills, the competent clinician will be able to optimize the dosage of a particular therapeutic in the course of routine clinical trials.

Mammalian species that may be treated with the present methods include canines and felines; equines; bovines; ovines; etc. and primates, particularly humans. Animal models, particularly small mammals, e.g. murine, lagomorpha, etc. may be used for experimental investigations.

### Kit

The present disclosure provides a kit with packaging material and one or more agents, compositions, or components described therein. A kit typically includes a label or packaging insert including a description of the components or instructions for use in vitro, in vivo or ex vivo, of the components therein. A kit can contain a collection of such components, e.g., the above-described cell populations, and optionally a second active agent such as a compound, therapeutic agent, drug or composition.

A kit refers to a physical structure that contains one or more components of the kit. Packaging material can maintain the components in a sterile manner and can be made of material commonly used for such purposes (e.g., paper, glass, plastic, foil, ampules, vials, tubes, etc).

A label or insert can include identifying information of one or more components therein, dose amounts, clinical pharmacology of the active ingredients(s) including mechanism of action, pharmacokinetics and pharmacodynamics. A label or insert can include information identifying manufacture, lot numbers, manufacture location and date, expiration dates. A label or insert can include information on a disease (e.g., an inherited or acquired or age-related disorder of myelin such as HD) for which a kit component may be used. A label or insert can include instructions for a clinician or subject for using one or more of the kit components in a method, use or treatment protocol or therapeutic regimen. Instructions can include dosage amounts, frequency of duration and instructions for practicing any of the methods, uses, treatment protocols or prophylactic or therapeutic regimens described herein.

A label or insert can include information on potential adverse side effects, complications or reaction, such as a warning to a subject or clinician regarding situations where it would not be appropriate to use a particular composition.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

### EXAMPLES

Human ESC (Genea19 line)-derived, mCherry-tagged glial progenitor cells (hGPCs) were prepared in the manner described in Wang et al., “Human iPSC-Derived Oligodendrocyte Progenitors Can Myelinate and Rescue a Mouse Model of Congenital Hypomyelination,” Cell Stem Cell 12(2):252-264 (2013), stored frozen at approximately 130 days in vitro (DIV), then thawed and cultured for an additional month to approximately 175 DIV. The cells were collected, spun and washed, concentrated to 100,000/μl, and then transplanted into neonatal human CSF-overexpressing host mice.

Human pre-microglial hematopoietic progenitor cells (hHPCs) were generated from the same Genea19 human ESC line, according to the microglial preparation method described in Abud et al., Neuron 94, 278-293, Apr. 19, 2017 but collected and frozen at the early hHPC stage, at 10 DIV.

Briefly, the microglial preparation was carried out using a differentiation base medium as described in Abud et al. The differentiation media consisted of a base media: phenol-free DMEM/F12 (1:1), insulin (0.02 mg/ml), holo-transferrin (0.011 mg/ml), sodium selenite (13.4 mg/ml) (can use ITS-G, 2% v/v, Thermo Fisher Scientific), B27 (2% v/v), N2 (0.5%, v/v), monothioglycerol (200 mM), Glutamax (1X), NEAA (1X), and additional insulin (5 mg/ml; Sigma) and filtered through a 0.22 mm filter. To prepare pre-microglial hematopoietic progenitor cells, at Day 0, isolated CD43⁺HPCs were washed using the iMGL base differentiation medium and centrifuged at 300×g for 6 min at room temperature. After centrifugation, supernatant was aspirated to waste and iHPCs were gently suspended in complete differentiation medium: M-CSF (25 ng/ml), IL-34 (100 ng/ml; PeproTech), and TGFβ-1 (50 ng/ml; Militenyi) added fresh each time. Cell density was adjusted to seed at 1-2×10⁵ cells in 2 mL of complete medium per well in growth factor-reduced Matrigelcoated 6-well plates. Every two days after that and until Day 10, each well was supplemented with 1 mL per well of complete differentiation medium. At Day 10, the cells were collected and frozen. At this 10 DIV point, the cells are pre-microglial hematopoietic progenitor cells, which are more amenable to transplant and post-transplant microglial differentiation. The cells were thawed on the day of injection and concentrated to 100,000/μl.

For generating dual chimeras (macroglial engraftment and microglial engraftment), Balb/c-Rag2^{-/-} x IL2gc^{-/-} x CSF1^{h/m} mouse hosts were used (Rathinam et al. Blood, 15 Sep. 2011, Volume 118, Number 11). For microglial engraftment alone, both Balb/c-Rag2^{-/-} x IL2gc^{-/-} x CSF1^{h/m} mice and NSG SGM3 x TMEM199CreER^{T2}; CSF1R^{loxP/loxP} mice were also used.

The hGPCs and hHPCs were mixed at the time of injection into a single vial at a 1:1 ratio, and then injected into 1 day-old (P1) neonatal immunodeficient NSG mice that had been crossed to human CSF1 overexpressing mice. The P1 mice were injected bilaterally at both anterior and posterior sites in the corpus callosum, as previously described (Windrem et al., “Neonatal Chimerization With Human Glial Progenitor Cells Can Both Remyelinate and Rescue the Otherwise Lethally Hypomyelinated Shiverer Mouse,” Cell Stem Cell 2:553-565 (2008); Wang et al., “Human iPSC-Derived Oligodendrocyte Progenitors Can Myelinate and Rescue a Mouse Model of Congenital Hypomyelination,” Cell Stem Cell 12(2):252-264 (2013); and Windrem et al., “A Competitive Advantage by Neonatally Engrafted Human Glial Progenitors Yields Mice Whose Brains Are Chimeric for Human Glia” The Journal of Neuroscience, Nov. 26, 2014, 34(48):16153-1616). Briefly, the mouse pups were first cryoanesthetized for cell delivery. Cells in 0.5 μl HBSS were then injected at each of 4 locations in the forebrain subcortex, specifically into the anterior and posterior anlagen of the corpus callosum bilaterally. These injections were delivered to a depth of 1.0 to 1.2 mm ventrally, depending on the weight/size of the pup (which varied from 1-1.5 g). All cells were injected through pulled glass pipettes, inserted directly through the skull into the presumptive target sites. The pups were then returned to their mother, until weaning at 21 days; at that point, each litter was moved to separate enriched housing. After weaning, mice were checked at least twice daily. Mice that died from immediate surgical complications or before weaning were excluded from the experiment. The cells were thus delivered as 4 separate injections of 50,000 cells each, with the injectates including a 1:1 ratio of hGPCs and hHPCs.

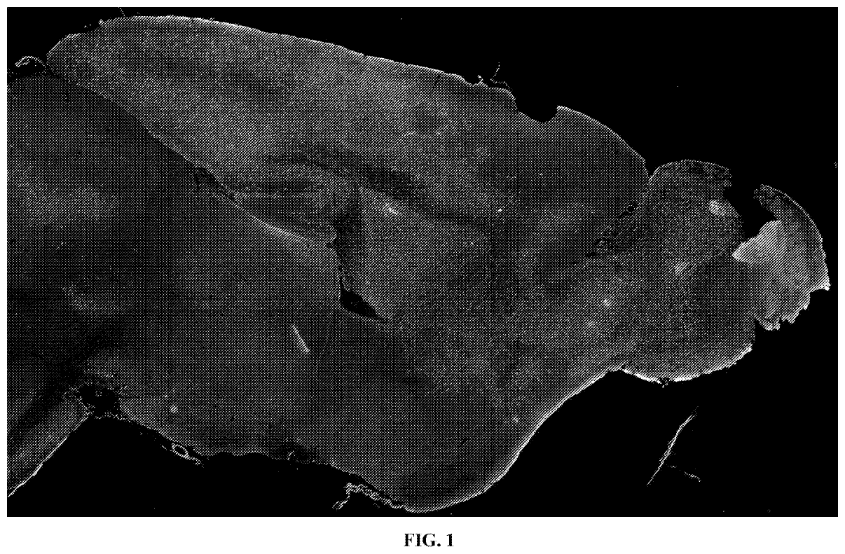

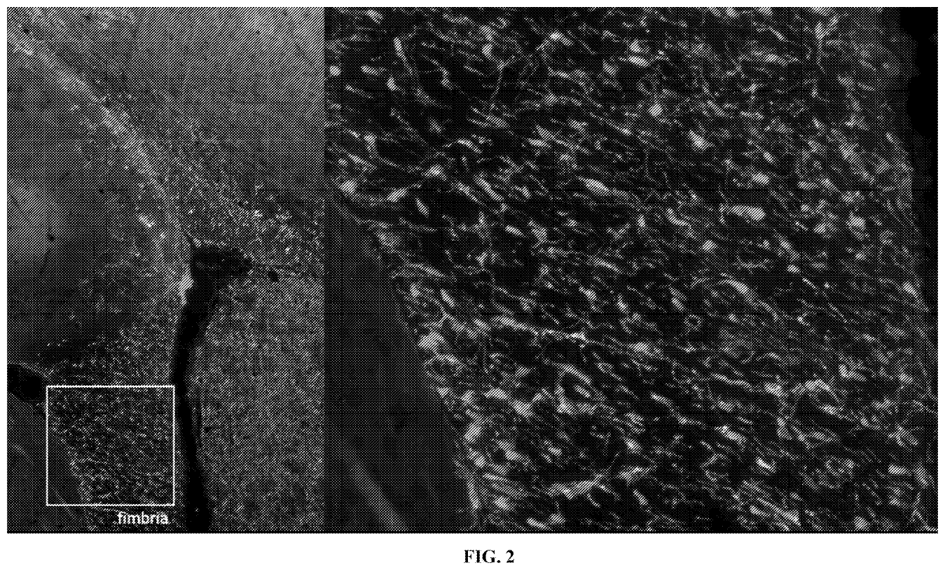

The mice were sacrificed at 8 weeks, revealing the widespread engraftment in the forebrain white matter of both human GPCs, hGPC-derived astrocytes, and hHPC-derived microglia, with large scale replacement of the corresponding mouse host cells. See FIGS. 1 and 2. To identify microglia cell, expression of purinergic receptor P2RY12 was examined by immune-fluorescence staining (green) while macroglial engraftments were observed via the mCherry red fluorescence.

The 200,000 combined total cell dose was selected as a starting point, based upon past experience with each cell type injected individually at that age. Both the doses and ratios of cell types can vary over a broad range of both doses (e.g., 100,000 to 600,000 total for mice) and ratios (e.g., 10:1 to 1:10).

The foregoing examples and description of the preferred embodiments should be taken as illustrating, rather than as limiting the present disclosure as defined by the claims. As will be readily appreciated, numerous variations and combinations of the features set forth above can be utilized without departing from the present disclosure as set forth in the claims. Such variations are not regarded as a departure from the scope of the disclosure, and all such variations are intended to be included within the scope of the following claims. All references cited herein are incorporated by reference in their entireties.

1. A composition suitable for treating glial-mediated human neurodegenerative disease or glial-mediated human neuropsychiatric disease comprising:
a population of isolated human microglial progenitor cells derived from pluripotent stem cells and
a population of isolated human macroglial progenitor cells derived from pluripotent stem cells.

2. The composition of claim 1, wherein the pluripotent stem cells are embryonic stem cells or induced pluripotent stem cells.

3. (canceled)

4. The composition of claim 1, wherein the numerical ratio of the human microglial progenitor cells to the human macroglial progenitor cells is about from 1:1,000 to 1,000 to 1.

5. (canceled)

6. The composition of claim 4, wherein the numerical ratio of the human microglial progenitor cells to the human macroglial progenitor cells is about 1:1.

7. A method of treating a glial-mediated neurodegenerative disease or a glial-mediated neuropsychiatric disease in a subject, said method comprising:
selecting a subject with a glial-mediated neurodegenerative disease or a glial mediated neuropsychiatric disease; and
introducing a population of isolated microglial progenitor cells derived from pluripotent stem cells and a population of isolated macroglial progenitor cells derived from pluripotent stem cells into the brain and/or brain stem of the selected subject to at least partially replace cells in the subject's brain with glial-mediated neurodegenerative disease or glial-mediated neuropsychiatric disease.

8. The method of claim 7, wherein the subject has a glial-mediated neurodegenerative disease selected from the group consisting of Alzheimer's disease, Lewy body disease, Multisystem atrophy, progressive supernuclear palsy, corticobasal degeneration, and Huntington's disease.

9. The method of claim 8, wherein the glial-mediated neurodegenerative disease is Alzheimer's disease.

10. The method of claim 7, wherein the subject has a glial-mediated neuropsychiatric disease, wherein the glial-mediated neuropsychiatric disease is schizophrenia.

11. The method of claim 7, wherein the selected subject is a human.

12. The method of claim 7, wherein the pluripotent stem cells are embryonic stem cells or induced pluripotent stem cells.

13. (canceled)

14. The method of claim 7, wherein said introducing is carried out by intraparenchymal, intracallosal, intraventricular, intrathecal, intracerebral, intracisternal, or intravenous transplantation.

15. The method of claim 7, wherein the population of isolated microglial progenitor cells and the population of isolated macroglial progenitor cells are from an autologous source or an allogenic source.

16. (canceled)

17. The method of claim 7, wherein said introducing is carried out by co-engrafting the population of isolated microglial progenitor cells and the population of isolated macroglial progenitor cells.

18. The method of claim 7, wherein the population of isolated microglial progenitor cells and the population of isolated macroglial progenitor cells are introduced separately and sequentially.

19. The method of claim 7, wherein the population of isolated microglial progenitor cells and the population of isolated macroglial progenitor cells are introduced simultaneously.

20. The method of claim 7, wherein the population of isolated microglial progenitor cells and the population of isolated macroglial progenitor cells are together in a composition.

21. The method of claim 7, wherein the ratio of the microglial progenitor cells to the macroglial progenitor cells is about from 1:1,000 to 1,000 to 1.

22. The method of claim 7, wherein each of the population of the microglial progenitor cells and the population of the macroglial progenitor cells are introduced to the subject at a dose of about 1×10² to about 1×10¹⁰.

23. A kit for treating glial-mediated human neurodegenerative disease or glial-mediated human neuropsychiatric disease comprising:
a population of isolated human microglial progenitor cells derived from pluripotent stem cells and
a population of isolated human macroglial progenitor cells derived from pluripotent stem cells.

24. The kit of claim 23, wherein the pluripotent stem cells are embryonic stem cells or induced pluripotent stem cells.

25. (canceled)
